(19) United States
(12) Patent Application Publication
(10) Pub. No.: US 2010/0022850 A1
(43) Pub. Date: Jan. 28, 2010

(54) TRANSDUCER ARRAY FOR SENSING PHYSIOLOGICAL INFORMATION

(75) Inventors: Gilbert W. Mc Kenna, Revere, MA (US); Lewis N. Harrold, Georgetown, MA (US)

Correspondence Address: DRIGGS, HOGG, DAUGHERTY & DEL ZOPPO CO., L.P.A., 38500 CHARDON ROAD, DEPT. DLBH, WILLOUGBY HILLS, OH, 44094, US

(73) Assignee: Analogic Corporation, Peabody, MA (US)

(21) Appl. No.: 12/181,024

(22) Filed: Jul. 28, 2008

(51) Int. Cl. A61B 5/00

(52) U.S. Cl. 600/301

(57) ABSTRACT

A physiologic parameter transducer array for a subject support includes at least one transducer configured for placement between the subject support and a subject being supported by the subject support. The at least one transducer senses an event indicative of a physiologic parameter of the subject that corresponds to the event. A signal processing device, in electrical communication with the at least one transducer, generates a signal indicative of the physiologic parameter. An identification component stores a unique identifier of the subject. The unique identifier associates the generated signal with the subject. A display component displays indicia indicative of the event.

### TECHNICAL FIELD

The following generally relates to and array of transducers, or transducer array, and finds particular application to sensing physiological information about a subject such as a human patient. However, the transducer array is also amenable to other applications in which it may be desirable to sense information about a subject or object.

### BACKGROUND

With some patients, vital signs are continuously monitored while they are in the hospital. For example, a patient in the intensive care unit (ICU) or coronary care unit (CCU) may be physically hooked up to physiological monitoring equipment that continuously and automatically measures and monitor heart rate, respiration rate, blood pressure, blood oxygen, etc. while they are in such a unit. With other patients, such equipment is hooked up to the patient only at the time of making such measurements and then removed from the patient. This may be done on a periodic basis. In yet other instances, rather than using such equipment to make such measurements, a nurse or other health care provider determines the patient's condition through observation.

In the latter case, the health care provider often has to physically interact with and observe the physical condition of the patient. The ambulatory or mobile nature of the patient may not be known. Such a patient may fall next to their bed or somewhere unobserved, without being found until someone enters their room. Also, as part of their treatment, if the patient is not moving in bed a health care provider may have to roll or otherwise turn the patient from one side to the other to help with blood flow and/or mitigate bed sores. For such a patient, it may be desirable to automatically monitor the patient's physiological information even though not requested or ordered by a treating physician. However, such equipment may not be available, or it may not be practical to continuously or periodically attach such equipment to a patient.

### SUMMARY

Aspects of the application address the above matters, and others.

In one aspect, a physiologic parameter transducer array for a subject support includes at least one transducer configured for placement between the subject support and a subject being supported by the subject support. The at least one transducer senses an event indicative of a physiologic parameter of the subject that corresponds to the event. A signal processing device, in electrical communication with the at least one transducer, generates a signal indicative of the physiologic parameter. An identification component stores a unique identifier of the subject. The unique identifier associates the generated signal with the subject. A display component displays indicia indicative of the event.

In another aspect, a physiologic parameter monitoring system includes a subject support and a subject support transducer array that includes at least one transducer that senses a physiologic state of a subject supported by the subject support. A signal processing device generates a signal indicative of the physiologic state, and an identification component stores a unique identifier of the subject. At least one monitoring device receives the signal. The at least one monitoring device includes a signal analyzer that obtains data representative of the physiologic state from the signal and associates the physiologic state with the subject based on the unique identifier.

In another aspect, a method includes sensing with a transducer of a transducer array at least one deflection indicative of at least one physiologic state of a subject supported by the transducer array, determining a physiologic state from the sensed deflection, and displaying the physiologic state on a presentation component of the transducer array.

Those skilled in the art will recognize still other aspects of the present application upon reading and understanding the attached description.

### BRIEF DESCRIPTION OF THE DRAWINGS

The application is illustrated by way of example and not limitation in the figures of the accompanying drawings, in which like references indicate similar elements and in which:

FIG. 1 illustrates an example physiologic parameter monitoring system;

FIGS. 2 and 3 illustrate example resistive based transducer arrays;

FIG. 4 illustrates an example inductive based transducer array;

FIGS. 5 and 6 illustrate example capacitive based transducer arrays;

FIG. 7 illustrates an example signal analyzer; and

FIG. 8 illustrates a method.

### DETAILED DESCRIPTION

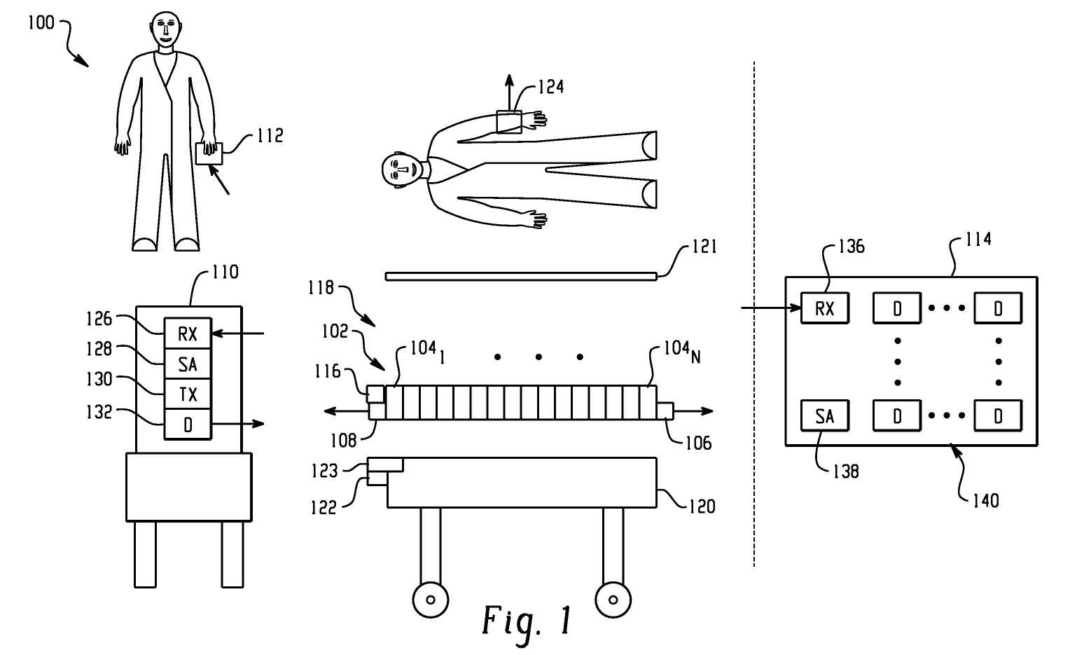

FIG. 1 illustrates a monitoring system 100 for monitoring a subject, such as a human. The system 100 includes a transducer array 102 with one or more transducers 104₁, . . . , 104_{N} (collectively 104), such as resistive, inductive, capacitive, acceleration, temperature, Polyvinylidene Fluoride (PVDF), piezoelectric and/or other transducers. One or more of the transducers 104 may be individually or aggregately activated and deactivated. The transducer array 102 is configured to sense deflection indicative of information about the subject, such as physiologic information like one or more physiologic parameters and/or states. Examples of such information include, but are not limited to, heart rate, respiration rate, respiration intensity, temperature, position, activity level, pressure points, blood flow, time on and off (use of) the transducer array 102, weight and wetness/enuresis. The transducer array 102 generates a signal indicative of the sensed information.

The transducer array 102 also includes an identification component 106 such as a radio frequency identification (RFID) tag, a bar code or the like that stores information indicative of the subject being monitored. Such information may include a unique identification for the subject and the transducer array 102. In one instance, the unique identification is used to verify that the sensed information is from the subject being monitored. For example, the identification component 106 may be configured to read or otherwise communicate with an identification tag such as a bracelet or badge worn by the subject. In this instance, the identification component 106 may first compare the subject identification stored therein with the subject identification from the bracelet. If the identification does not match, the transducer array 102 may sense and store information along with indicia noting that subject validation failed. In another instance, the unique identification is included in or provided with the generated signal. In this instance, the unique identification can be used to map the signal and/or the content thereof to the corresponding subject, for example, at a remote monitoring station.

A signal processing device 108 is in electrical communication with the sensing array 102 and receives the signal generated thereby. The sampling rate of the signal processing device 108 is configured to sample the signal, which, in one instance ranges from .05 Hz to about 300 Hz, at the suitable sampling rate, such as around the Nyquist rate. The signal processing device 108 processes the signal and provides the processed signal to a local monitoring system such as a bed side monitor 110, a portable monitoring device 112, a remote monitoring system such as a central monitoring station 114, and/or other system. The signal processing device 108 may include various signal processing components such as a signal amplifier, a signal conditioner, a filter, an analog to digital converter, a digital to analog converter, a signal encoder, a signal encryptor, a radio frequency (RF) link, and/or one or more other processing components. The signal processing device 108 may also include memory that can store the sampled signal, for example, for at least one 8 hour shift. A display 116 presents the subject information in real-time or when recalled by a health care provider. Such information may include numerical values and/or other indicia indicative of normal and out of bound events.

In the illustrated embodiment, the transducer array 102 is part of a layer 118 that is positioned with respect to a patient support such as a bed 120. The layer 118 includes a material that is flexible, and thus can stretch and flex in one or more directions to accommodate different patient supports, and can be affixed, integrated with and/or conform to one or more of the beds 120. The layer 118 is also re-usable, portable, modular, expandable, and cleanable, and may be cleaned and re-used with a number of different subjects. The layer 118 is positioned with respect to the bed 120 so that at least one of the transducers 104 is located between a subject sitting or lying on the bed 120 and the bed 120. A cover 121 such as a bed sheet may be placed between the subject and the layer 118. The cover 121 provides a barrier between the subject and the transducer array 120. The embodiment may be reusable or single patient and disposable.

It is to be appreciated that the bed 120 may be located in a patient care facility such as a hospital, the patient's home, or other location. The illustrated bed 120 is mobile in that it includes wheels and can be moved around a room and to other locations in the patient care facility. The illustrated bed 120 includes a receiver 122 that receives the sensed information and a display 123 for displaying the sensed information such as a visual display. In another instance, the bed 120 does not include the receiver 122 or display 123. In other embodiments, the support includes a chair, a wheel chair, a transportation cart, or other support, including a support which is part of another system such as a table of an imaging system.

A wearable transducer 124 such as a wristlet, anklet, or the like is worn by the subject. The wearable transducer 124 is configured to sense information similar to the information sensed by the transducer 104 and communicating with the signal processing device 108. This allows the subject to be monitored when the subject is out of the bed 120 or in a position on the bed 120 at which it may be difficult to otherwise obtain a suitable signal, for example, one with a desired signal-to-noise ratio. The transducer 124 may include a wireless transmitter for wireless communication and/or an electrical contact for wired communication. The wearable transducer 124 may also include an identification component, such as an RFID tag, that stores information indicative of the subject. As such, the wearable transducer 124 may validate the subject and tag subject information as discussed above. In one instance, the wearable transducer 124 includes relatively low power circuitry and incorporates a small, light-weight battery(s) to power a transducer and transmitter of the wearable transducer 124. The wearable transducer 124 can be omitted.

The bed side monitor 110 generally is used to monitor information about one patient at a time. A receiver (RX) 126 is configured to receive at least the signal from the signal processing device 108. A signal analyzer (SA) 128 processes the signal. In one instance, the signal analyzer 128 includes software that when executed extracts desired information from the signal, including physiologic and identification information. This may include extracting information about at least one physiological parameter such as heart rate, respiration rate, respiration intensity, temperature, position, blood flow, activity, and wetness from the signal. In another instance, the signal analyzer 128 removes or filters undesired information such as noise from the signal. Noise as used herein means both common electrical noise, such as from fluorescent light fixtures as well as signals whose origin is non-physiological. A display (D) 132 displays the extracted information in a human readable format. This may include displaying wave forms and/or numerical values. The bed side monitor 110 may also include notification capabilities such as visual and/or audible indicators for alarms or warnings. A transmitter (TX) 130 is configured to convey information from the bed side monitor 110, for example, to the central monitoring station 114. Communication can be through a wired, wireless or other suitable communications interface.

As illustrated, the portable monitoring device 112 is capable of wirelessly communicating with the signal processing device 108. It generally is used to obtain substantially instantaneous snap shots of information such as once an hour, every four hours, etc. as, for example, determined by a physician or other health care personnel. Such snap shots may be obtained as a nurse assesses the subject being cared for, for example, at the beginning of a shift while obtaining and manually recording vital signs. As such, the portable monitoring device 112 often is a hand-held battery powered device that is activated to obtain the information and then turned off once the information is recorded. A docking station holds the portable monitoring device 112 when not in use. The docking station may be used to charge a rechargeable battery of the portable monitoring device 112.

The central monitoring station 114 generally is part of a larger scale monitoring system for concurrently monitoring a plurality of subjects. A receiver 136 is configured to receive a signal from the signal processing device 108 and/or wearable transducer 124. In one instance, the central monitoring station 114 continuously polls multiple signal processing devices 108 and/or wearable transducers 124, and continuously records the data. The identification information from the identification component 106 facilitates identifying and mapping the signal to the corresponding patient at the central monitoring station 114. A signal analyzer 138 processes the signal. The signal analyzer 138 extracts desired information from the signal, such as at least one of heart rate, respiration rate, respiration intensity, temperature, position, blood flow, activity, and wetness from the signal and/or removes or filters undesired information from the signal such as noise. The central monitoring station 114 may store information and/or transfer information to a central storage or other repository.

One of the displays 140 displays the extracted information for a corresponding patient in a human readable format. The central monitoring station 114 also includes notification capabilities such as visual and/or audible indicators for alarms or warnings. In one instance, the central monitoring station 114 incorporates an intuitive display with status lights (e.g. colored LED's) that allows a user to concurrently see the relative status of the patients. A change of color in a status light for a particular patient could indicate a reportable change in a parameter, as pre-programmed by the user, for example, from green to yellow or yellow to red. A flashing light or area on the monitor display may indicate a sudden or severe out-of-bounds condition. An audible alarm could also be set to complement the flashing status light to alert the user of the sudden/severe change in physiological data or trends in data which indicate a change in physiological status. When a patient's status changes, a second level of data could be automatically displayed on a secondary screen (e.g. a computer monitor) to identify the patient, location, plots of the historic trend data for the physiological parameters, and type of change (i.e. heart rate, respiration rate, temperature, etc). A measured parameter for an individual patient can be customized for normal and out-of-bounds conditions depending on the patient's ailment and/or condition.

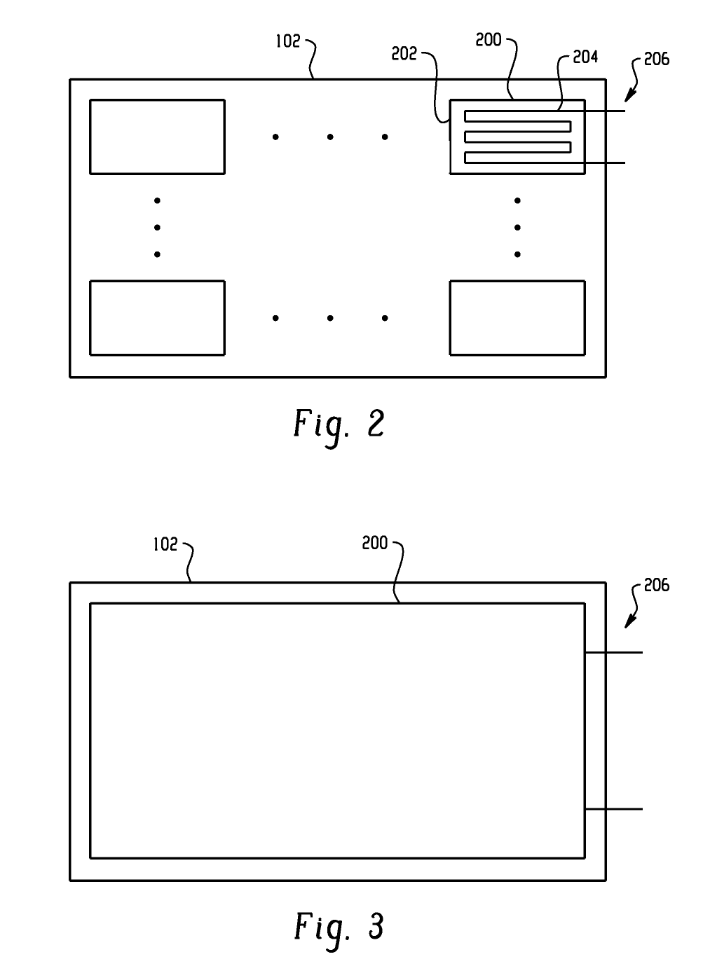

As briefly noted above, various types of transducers 104 can be used, including a plurality of different types of transducer technologies. Examples of such transducers include, but are not limited to, resistive, inductive, capacitive, PVDF, piezoelectric, accelerometric, and temperature. As such, the transducers 104 can sense deflections or events to the transducer array 102 resulting from the heart beating, breathing, blood flow and patient movement, liquids on the transducer array 102 from incontinence and urine or blood spills, temperature, and/or other physiological information. FIGS. 2-6 illustrate non-limiting examples of the transducer array 102 with different types of transducers 104. Note that in some instance, the transducers 104 may be affixed to a sub-portion of the transducer array 102, such as a sub-portion of a surface of the transducer array 102, integrated within a depth or thickness of the transducer array 102, and/or otherwise coupled to the transducer array 102.

Initially referring to FIG. 2, which shows a view looking down into the bed 120, the transducer array 102 is illustrated with at least one resistive based transducers 200 disposed thereon. The transducer 200 includes a backing 202 with at least one conductive strip 204. In this example, each transducer 200 occupies a sub-set of the transducer array 102. In other instances, as shown in FIG. 3, a single transducer 200 may occupy a substantial portion of the transducer array 102. The backing 202 generally is flexible and insulating, and the conductive strip 204 is formed in a suitable pattern, such as a meandering pattern as shown. In one instance, the conductive strip 204 is affixed to the backing 202 via a suitable adhesive. In another instance, a conductive layer is formed on the backing 202 and the conductive strip 204 is formed through masking and etching. Other techniques for attaching and/or forming the conductive strip 204 are also contemplated herein.

The conductive strip 204 includes copper, gold, silver and/or one or more other conductive materials, and has a known electrical characteristic such as a known resistance. When the conductive strip 204 is deformed or deflected (e.g., compressed or stretched), the electrical resistance changes in a known and defined manner, with a magnitude being proportional to the deflection. As such, a deflection induced by the patient (e.g., heart beating, breathing, temperature, etc.) causes a change in resistance indicative of the event. The transducer 200 produces an output signal that is indicative of the resistance, and the resistance is readable or measureable from terminals 206. The resistance may also change as a consequence of the presence of a liquid on the transducer 200. Liquids such as urea generally are more conductive than liquids like water and, thus, can be characterized through resistance. In instances in which there are more than one of the transducers 200, two or more of the transducers 200 can be interconnected to form a matrix, grid or network of such transducers 200.

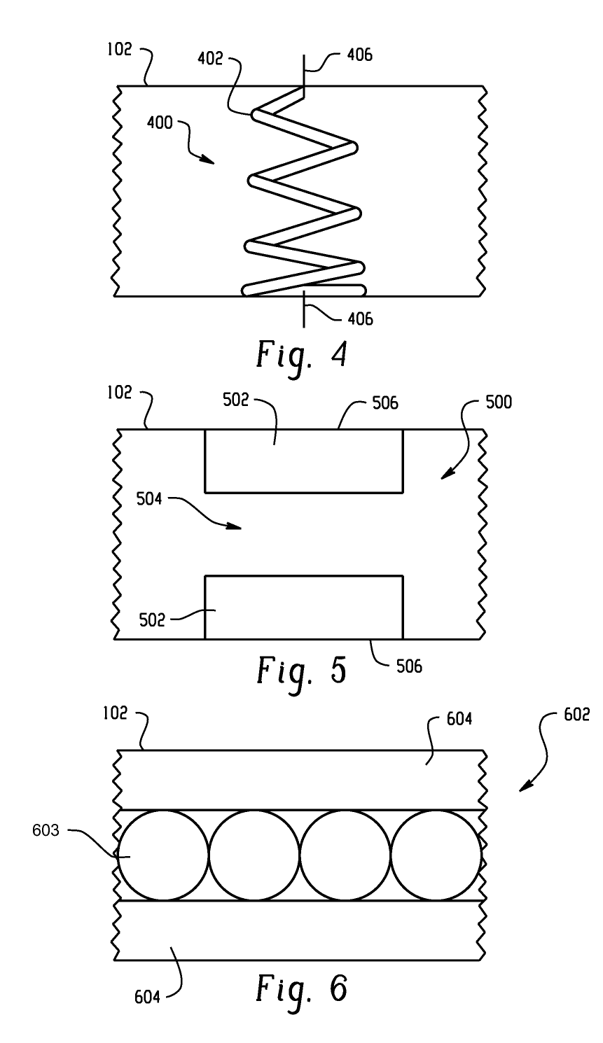

Turning to FIG. 4, a portion of a side view of the transducer array 102 is illustrated with at least one inductive based transducer 400. In the illustrated instance, the transducer 400 includes a coil 402, which in this example, is wound into a spiral and includes a plurality of windings. In other embodiments, the coil 402 may be shaped otherwise, for example, like a helical or other shape. The inductance of the coil 402 is measurable and inversely proportional to the spacing between the windings or turns. A transducer array deflection induced by the patient (e.g., heart beating, breathing, temperature, etc.) causes a change in the length of the coil 402, which changes the inductance of the coil 402. The magnitude of the inductance of the coil 402 is proportional to the deflection, and is readable or measurable at terminals 406. Similarly, where more than one of the transducers 400 are inductor based, the transducers 400 can be interconnected to form a network of such transducers 400.

In FIG. 5, the transducer array 102 includes at least one capacitive based transducer 500. Such a transducer 500 includes a pair of flexible conductors or plates 502 separated by a dielectric 504 such as air, which inhibits charge deposited on the conductors 502 from moving between the plates 502. The capacitance of the transducer 500 is a function of the distance between the plates 502 and the characteristics of the dielectric. A transducer array deflection induced by the patient (e.g., heart beating, breathing, etc.) causing a change in the distance between the plates 502, changes the capacitance, and the magnitude of the capacitance is proportional to the change in plate distance caused by the deflection. The characteristics of the dielectric may change due to a liquid on the sensing array 102. As such, a change in the characteristics of the dielectric induced by the patient (e.g., incontinence, spills, etc.), changes the capacitance. Again, the capacitance is measureable and readable from the terminals 506. FIG. 6 illustrates an example in which a dielectric 602 includes a plurality of plenum chambers 504 such as air filled bladders or other plenum chambers, sandwiched between plates 604, which include conductive films, such as aluminum or other metallic films. A capacitance can be read from the films for each of the plurality of air filled chambers 604.

Additionally or alternatively, the transducer 104 may include a component that measures acceleration or gravity induced reaction forces such as inclination, vibration and shock, such as a single or multi axis accelerometer or the like. In one instance, such a component includes a cantilever beam with a seismic mass and deflection sensing circuitry. Under the influence of gravity or acceleration such as that caused by a beating heart, breathing, blood flow, movement, temperature, etc. the mass deflects, and the deflection is measured.

Additionally or alternatively, the transducer 104 may include a component such as a Polyvinylidene Fluoride (PVDF) component such as a film that produces signals proportional to bending or stretching. In one instance, such a produces a charge when bent or flexed, and the charge is proportional to the amount of bend or flex that occurred. The PVDF component may come in sheet form or otherwise, and can be chemically machined like printed circuit boards. A suitable PVDF component includes a poly film with two conductive surfaces deposited thereon, for example, one on each side, and electronics soldered or electrically bonded thereto so that the circuits are integrated therewith or built in.

Additionally or alternatively, the transducer 104 may include a thermocouple, a thermister, or other temperature sensing component. Additionally or alternatively, the transducer 104 may include weigh scale transducer or other weight sensing component.

It is to be understood that the above depictions, examples, and descriptions are for illustrative purposes and are non-limiting, and other types of transducers are also contemplated herein.

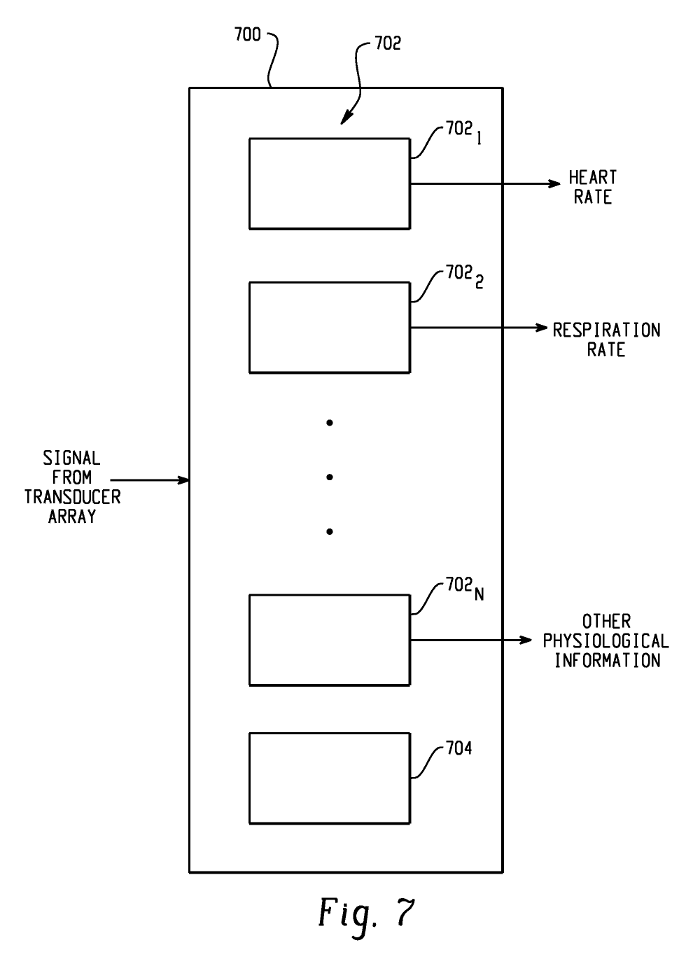

FIG. 7 illustrates an example signal analyzer 700, which can be used in connection with one or more of the monitors 110, 112 and 114. The illustrated signal analyzer 700 analyzes the signal indicative of the sensed physiological information through signature analysis. In one instance, the signal is analyzed based on a repetitive nature of the desired signal. The physical location of the corresponding transducer 104 on the transducer array 102 in relation to the subject combined with the relative power spectrum of the signal may further facilitate distinguishing between the signal and noise. Generally, the signal analyzer 600 may be configured for relatively larger scale, non-repetitive motion, such as patient movement, and/or smaller scale, substantially repetitive motion, such as breathing or blood flow pulsing at the heart rate. A rule based or other feature extractor approach such as an explicitly and/or implicitly trained classifier or power spectrum may be used.

The signal analyzer 700 includes a plurality of signal sensing components 702, each tuned to sense particular physiological information in the signal. By way of example, the signal sensing component 702₁ may be tuned to extract heart rate related information from the signal. Generally, heart rates may vary from about 30-300 beats or cycles per minute, which is about 0.5-5 Hertz (Hz). As such, the signal sensing component 702₁ may include a band pass filter that passes only signals with a frequency within a frequency range of 0.5-5 Hz and filters signals with a frequency outside of this range, such as 60 Hz noise from an alternating current (AC) line. By way of another example, the signal sensing component 702₂ may be tuned extract respiration rate, which, generally, may vary from about 3-150 cycles per minute, or 0.05-2.5 Hz. As such, the signal sensing component 702₂ may include a band pass filter that passes signals within a frequency range of .05-2.5 Hz. A Fourier analysis of the signal, and determination of the relative power spectrum of the associated frequencies, can further aid the determination of the heart rate from the composite signal from the array.

Heart beats and respirations generally come from the chest area, so location provides further information for distinguishing between information corresponding to heart and respiration rate and other signals such as noise or other physiological information. For instance, a signal with a frequency within the above heart rate and respiration rate range, but coming from the feet of the subject may not be deemed valid physiological information. Likewise, the power spectrum of the signal may be used as a feature signature to further distinguish a desired signal from an extraneous signal. A fast Fourier transform or other technique may be used to determine the power spectrum. The relative intensity of the signal may also be used to determine such information such as whether the patient is lying on their stomach or back.

A signal sensing component 702 for wetness/enuresis would look for a different pattern than that of the above repetitive signals, being positional and time related, for example, more central initially, and then spreading out in a slow wave front from that central location. The pattern would also tend to be lower frequency relative to breathing, being more like a direct current (DC) change over a period of minutes.

A noise cancellation component 704 determines a noise signal and can be use the signal to tare out residual background, non-physiologic signals which are repetitive. This may facilitate identifying and extracting desired physiologic information. The noise cancellation component 704 can be omitted.

Variations and alternatives are discussed.

In the illustrated embodiment, the signal processing device 108 is part of the sensing array 102. In another embodiment, the signal processing device 108 is a separate component, electrically coupled to the sensing array 102. In yet another embodiment, the signal processing device 108 is part of the bed 120. In still another embodiment, the signal processing device 108 is part of one or more of the monitors 110, 112, and 114.

In another embodiment, at least one of the transducers 104 includes an RF transmitter or infrared emitter, and broadcasts or otherwise conveys the sensed information.

In another embodiment, an air, foam, or other material layer is placed between the transducer array 102 and the bed 120. Such a layer may facilitate dampening signals generated by events other than the event indicative of the physiologic parameter such as noise transferred through the bed 120 to the sensing array 102 like 60 Hz from an AC power source or other noise sources. In one instance, the layer includes a plurality layers of materials selected to individually or aggregately act as a signal dampening component between the transducer array 102 and the subject.

In another embodiment, the transducer array 102 is laminated or otherwise include a protective barrier.

In yet another embodiment, the transducer array 102 is part of the bed 120.

In another embodiment, one or more of the transducers 104 are located in/on the transducer array 102 based on signals being sensed. For example, in one instance a first plurality of cardiac or respiratory transducers are located by the thoracic region, a second plurality of moisture transducers are located by different regions, etc.

In another embodiment, two or more of the transducer arrays 102 are concurrently used with a patient.

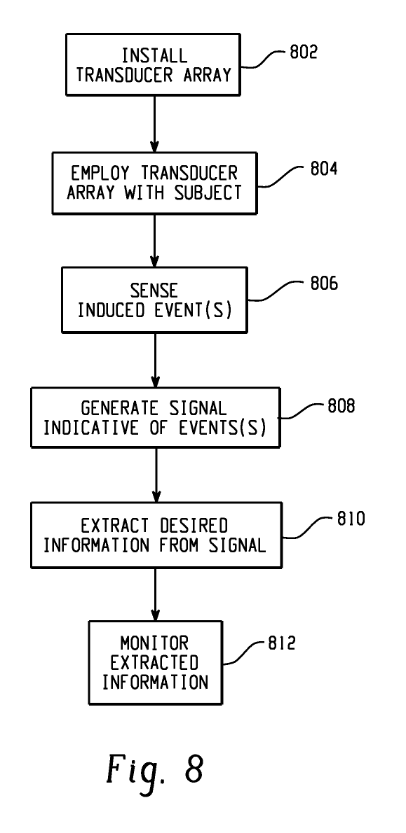

FIG. 8 illustrates a method for determining physiological information. At 802, the sensing array 102 is placed on a patient support such as the bed 120. A sheet or other cover may be placed over the sensing array 102. At 804, the patient contacts the sensing array 102. The contact may be physical contact or indirect contact, for example, urine, sweat, water, etc. At 806, the sensing array 102 senses the event(s). At 808, the sensing array 102 generates a signal indicative of the sensed contact. At 810, physiological information is extracted from the signal. At 812, the physiological information is monitored to identify a state of the patient.

The application has been described with reference to various embodiments. Modifications and alterations will occur to others upon reading the application. It is intended that the invention be construed as including all such modifications and alterations, including insofar as they come within the scope of the appended claims and the equivalents thereof.

1. A physiologic parameter transducer array for a subject support, comprising:
at least one transducer configured for placement between the subject support and a subject being supported by the subject support, wherein the at least one transducer senses an event indicative of a physiologic parameter of the subject that corresponds to the event;
a signal processing device, in electrical communication with the at least one transducer, that generates a signal indicative of the physiologic parameter;
an identification component that stores a unique identifier of the subject, wherein the unique identifier associates the generated signal with the subject; and
a display component that displays indicia indicative of the event.

2. The transducer array of claim 1, wherein the physiologic parameter includes one of heart rate, respiration rate, respiration intensity, temperature, position, a pressure point, activity, weight and wetness

3. The transducer array of claim 1, wherein the event results in a change in an electrical characteristic of the transducer array.

4. The transducer array of claim 3, wherein the transducer array includes a coil, and the electrical characteristic is an inductance of the coil which changes as a function of the a change in a length of the coil in response to a deflection induced by a subject.

5. The transducer array of claim 3, wherein the electrical characteristic is a resistance or a capacitance of the transducer array.

6. The transducer array of claim 1, wherein the identification component includes a radio frequency identification (RFID) tag, and the unique identifier is stored in and obtained from the RFID tag

7. The transducer array of claim 6, wherein the signal processor employs the unique identifier to determine if the event corresponds to the subject

8. The transducer array of claim 1, wherein the at least one transducer includes an accelerometric transducer.

9. The transducer array of claim 1, wherein the at least one transducer includes a coil with an inductance that is a function of a length of the coil, and the length of the coil is a function of the event.

10. The transducer array of claim 1, wherein at least one of the signal processing devices and the at least one transducer forms a part of the subject support.

11. The transducer array of claim 1, further including a signal dampening component disposed between the at least one transducer and the subject support, wherein the dampening component dampens signals generated by events other than the event indicative of the physiologic parameter.

12. The transducer array of claim 1, wherein the transducer array includes a material that is flexible, and transducer array flexes and stretches to accommodate different subject supports.

13. The transducer array of claim 1, wherein the at least one transducer includes at least two electrically conductive layers with a plenum chamber disposed therebetween so as to form a capacitor.

14. The transducer array of claim 13, wherein the event causes a distance between the layers to change, thereby changing a capacitance associated with the transducer.

15. The transducer array of claim 1, wherein the at least one transducer includes an accelerometer, and the event is an accelerometric reaction force

16. The transducer array of claim 1, wherein the event corresponds to a temperature of the subject.

17. The transducer array of claim 1, further including a signal analyzer that extracts data indicative of the physiologic parameter from the signal.

18. The transducer array of claim 17, wherein signal analyzer extracts the data based on a repetitive nature of the parameter.

19. The transducer array of claim 17, wherein signal analyzer extracts the data based on a location on the transducer array where the event is sensed.

20. The transducer array of claim 17, wherein signal analyzer extracts the data based on a power spectrum of the signal

21. A physiologic parameter monitoring system, comprising:
a subject support;
a subject support transducer array that includes at least one transducer that senses a physiologic state of a subject supported by the subject support;
a signal processing device that generates a signal indicative of the physiologic state;
an identification component that stores a unique identifier of the subject; and
at least one monitoring device that receives the signal, the at least one monitoring device including a signal analyzer that obtains data representative of the physiologic state from the signal, wherein the at least one monitoring device associates the physiologic state with the subject based on the unique identifier.

22. The system of claim 21, wherein the transducer includes a coil with windings and has an inductance value that is a function of a change in a distance between the coil windings corresponding a change in the physiologic state.

23. The system of claim 21, wherein the transducer array includes one or more of a resistive, an inductive, a capacitive, an accelerometric, a temperature, a Polyvinylidene Fluoride, or a piezoelectric transducer.

24. The system of claim 21, wherein the subject support includes a display that presents indicia indicative of the sensed physiologic state.

25. The system of claim 21, further including material disposed between the subject support and the subject support transducer array or part of the subject support transducer array, wherein the material dampens signals generated indicative of information other than physiologic states of the subject.

26. The system of claim 21, wherein the signal analyzer obtains the data based on all of a repetitive nature of the state, a location where the state is sensed, and power spectrum of the signal

27. The system of claim 21, wherein the transducer array includes a first set of transducers configured to sense a first physiological signal and located by a first region of the subject that produces the first physiological signal, and a second set of transducers configured to sense a second physiological signal and located by a second region of the subject that produces the second physiological signal.

28. The system of claim 21, further including at least a second transducer array 102, wherein the first and second transducer arrays are concurrently used to sense the physiologic state of the subject.

29. A method, comprising:
sensing with a coil of a transducer array at least one deflection indicative of at least one physiologic state of a subject supported by the transducer array, wherein the deflection changes a length of the coil and an inductance value of the transducer coil;
determining a physiologic state from the change in the inductance value; and
displaying the physiologic state on a presentation component of the transducer array.

30. The method of claim 29, further including
generating a signal indicative of the physiologic state;
obtaining a unique identifier of the subject; and
transmitting the signal and the unique identifier to a remote physiologic state monitoring system.

31. The method of claim 29, further including:
extracting the physiologic information from the signal; and
displaying the extracted physiologic information with indicia identifying the subject.
